(19) United States
(12) Patent Application Publication
(10) Pub. No.: US 2023/0397877 A1
(43) Pub. Date: Dec. 14, 2023

(54) SWALLOWING CAPTURE AND REMOTE ANALYSIS SYSTEMS INCLUDING MOTION CAPTURE

(71) Applicant: SWALLIS MÉDICAL, COLOMIERS (FR)

(72) Inventors: Nicolas PERRIN, KOLBSHEIM (FR); Linda NICOLINI, TOULOUSE (FR); Fabrice NEVEU, MONTPELLIER (FR)

(21) Appl. No.: 18/246,983

(22) PCT Filed: Sep. 22, 2021
(86) PCT No.: PCT/EP2021/076097

§ 371 Date: Mar. 31, 2023

(30) Foreign Application Priority Data
Sep. 29, 2020 (EP) 20198909.2

(51) Int. Cl. A61B 5/00; A61B 5/11; A61B 5/0205

(52) U.S. Cl. CPC A61B 5/4205; A61B 5/1128; A61B 5/02055; A61B 5/0002; A61B 5/7264; A61B 5/742; A61B 5/1127; A61B 2562/0204

(57) ABSTRACT

A system for capturing the swallowing of at least one user, includes at least one swallowing detection device including at least one swallowing detection sensor which is configured to acquire at least one swallowing signal of the user, at least one system for capturing movements of the user, which system is configured to acquire at least one movement signal of the user during the capture of the at least one swallowing signal of the user by the sensor of the swallowing detection device.

### TECHNICAL FIELD OF THE INVENTION

The technical field of the invention is that of swallowing detection.

The present invention relates to a system for sensing swallowing and remotely analysing swallowing comprising motion capture, and in particular motion capture using at least one camera.

### TECHNOLOGICAL BACKGROUND OF THE INVENTION

Patients with swallowing disorders, also known as “dysphagia”, present difficulties in swallowing food, for example the lack of coordination in the path of food from the mouth to the stomach via the pharynx and oesophagus, and risks of false passages (pulmonary aspirations). With regard to false passages, the term penetration is used if the food enters the larynx but remains above the glottis, the vocal cords, and aspiration if it passes the vocal cords. Aspiration causes one of the body's responses, normally cough, but if there is a sensitivity disorder, aspiration may be cough-free and therefore silent.

Dysphagia can occur, for example, in patients after a stroke, after a traumatic brain injury, after amyotrophic lateral sclerosis or in patients with Alzheimer's disease or neurodegenerative diseases.

In order to detect the level of dysphagia in a patient, for example to evaluate the risk of false passages, the type, cause and/or severity of dysphagia, numerous examination methods have been developed.

In particular, the study of swallowing accelerometery has been developed in response to a need for non-invasive methods of evaluating dysphagia, particularly with the miniaturisation and improved accuracy of electronic accelerometers. Collar devices including an accelerometer have been developed for the acquisition of swallowing accelerometery signals at the larynx. Indeed, swallowing can be divided into three phases:
the oral phase of swallowing is voluntary and comprises the posterior motion of the tongue and the hyoid bone,
the laryngopharyngeal phase is automatic and reflexive and includes laryngeal motion, elevation of the hyoid bone, closure of the epiglottis and passage of the bolus to the oesophageal orifice, and
the oesophageal phase is reflexive and comprises peristaltic contraction, repositioning of the hyoid bone and larynx and reopening of the epiglottis.

Sensing these signals allows them to be processed by a computer and characterised in relation to these three phases of swallowing, and automatic classification techniques have thus been applied to these signals in prior art for the detection of dysphagia, aspiration, silent false passages and/or for the evaluation of the level of dysphagia.

Such collar devices may further comprise a laryngeal sound sensor, for example a microphone, for the measurement of swallowing sound, for example in order to filter and analyse breath sounds, cough sounds and sounds related to the patient's voice. The microphone may also allow the detection of swallowing, for example by detecting the laryngeal rise sound corresponding to the rise of the larynx when the bolus is located in the oropharynx and/or hypopharynx, the upper sphincter opening sound corresponding to the transit of the bolus through the upper sphincter, and the laryngeal release sound corresponding to the descent and opening of the larynx when the bolus has reached the oesophagus, as described by Sylvain Morinière et al. in “Origin of the Sound Components During Pharyngeal Swallowing in Normal Subjects”, Dysphagia, 2008.

These collar devices can be used for the detection and analysis of swallowing in a patient with dysphagia, or for the analysis of swallowing rehabilitation exercises. In swallowing detection and analysis, a user wears a necklace device for which signals are analysed generally by a device connected to the necklace such as a smartphone or other specialised electronic device. In order to analyse the data and carry out exercises, patients using such devices should still visit a specialist, particularly as the data collected by the sensor alone does not allow for a precise and complete analysis of swallowing or for feedback to the user on their swallowing behaviour in order to suggest possible exercises. Furthermore, although signals measured by the accelerometers and/or microphones allow characterisation of swallowing, this characterisation does not allow to trace the origin of the swallowing problem, that is its cause, but only to indicate whether the swallowing was right or wrong.

There is therefore a need to be able to study a patient's swallowing in a complete and accurate manner.

### SUMMARY OF THE INVENTION

The invention offers a solution to the previously discussed problems, by allowing a complete and accurate study of swallowing, allowing rehabilitation, not requiring a trip to the practitioner at the time of measurement of signals by a detection device.

One aspect of the invention relates to a system for sensing swallowing of at least one user, the system being characterised in that it comprises:
At least one swallowing detection device comprising at least one swallowing detection sensor configured to acquire at least one user swallowing signal,
At least one user motion capture system configured to acquire at least one user motion signal upon sensing the at least one user swallowing signal by the swallowing detection device sensor.

By virtue of the invention, it is possible to study the entire swallowing and to have an objective external view of swallowing. It is thus possible, by virtue of the presence of a user motion capture system, to associate one or more motions of the user with swallowing measured by a swallowing detection sensor, and thus to trace a potential cause of wrong swallowing, which may be wrong posture or wrong motion.

By jointly measuring swallowing by an accelerometer or microphone at the larynx and motions by a user motion capture system, swallowing sensing is enriched and it is possible to trace the cause of wrong swallowing and to analyse a posteriori motions of the user that led to the result of the swallowing characterisation.

In addition to the characteristics just discussed in the preceding paragraph, the sensing system according to one aspect of the invention may have one or several additional characteristics among the following, considered individually or according to any technically possible combinations:
the motion capture system comprises at least one camera,
the motion capture system further comprises at least one motion capture marker in cooperation with the at least one camera,
the at least one camera is configured to acquire images of the user's head, neck and chest,
the swallowing sensing system further comprises two cameras, one of the two cameras being configured to acquire frontal images of the user and another of the two cameras being configured to obtain profile images of the user,
the swallowing detection sensor is a microphone to detect swallowing sound and/or an accelerometer to detect swallowing vibration,
the swallowing detection device further comprises at least one sensor from heart rate, body temperature, sweating, breath sound, respiratory rate and muscle activity sensors,
the swallowing sensing system further comprises a network module configured to send through a network the at least one user swallowing signal and the at least one user motion signal upon sensing the at least one user swallowing signal,
the swallowing sensing system further comprises a processor configured to characterise the user's swallowing from the user swallowing signal and the user motion signal.

Another aspect of the invention relates to a system for analysing swallowing of at least one user comprising:
the user swallowing sensing system according to the invention and
a remote analysis device comprising:
   a network module configured to receive from the network module of the sensing system, via the network, the at least one user swallowing signal and the at least one user motion signal upon sensing the at least one user swallowing signal and
   a display module configured to simultaneously display the at least one user swallowing signal and the at least one user motion signal upon sensing the at least one user swallowing signal.

In addition to the characteristics just discussed in the preceding paragraph, the analysis system according to one aspect of the invention may have one or several additional characteristics among the following, considered individually or according to any technically possible combinations:
the remote analysis device comprises a processor configured to characterise the user's swallowing from the user swallowing signal,
characterising swallowing by the processor includes classifying the swallowing signal,
the processor is configured to characterise the user's swallowing by further using the user motion signal.

The analysis system according to the invention avoids the need for patients to travel to their practitioner to analyse their swallowing. This also allows live swallowing analyses, by having access to all the information necessary for the remote swallowing analysis: access to swallowing signal measurement information by virtue of the swallowing detection sensors as well as access to visual information by virtue of the cameras, with access to this information being simultaneous by virtue of the display module of the analysis system. This simultaneous display makes it possible to link both types of information and to work remotely as if the user were physically in the same room as the practitioner.

In addition, characterising swallowing can be performed remotely by the remote analysis device, allowing the cost of swallowing detection devices to be reduced by reducing the need for computing power at the swallowing detection devices and shifting the computing power to a remote device, accessible and usable by a practitioner for example.

Yet another aspect of the invention relates to a method for sensing swallowing of at least one user by a swallowing sensing system according to the invention, the method comprising the steps of:
Sensing at least one swallowing signal by a swallowing detection device of the swallowing sensing system;
Acquiring, by the motion capture system, the user motion signal simultaneously with sensing the swallowing signal by the swallowing detection device.

Still another aspect of the invention relates to a method for analysing swallowing of at least one user by a swallowing analysis system according to the invention, the method comprising the steps of:
Receiving, by the remote analysis device, the at least one swallowing signal and the at least one user motion signal acquired simultaneously with sensing the swallowing signal;
characterising swallowing by the processor of the swallowing analysis system from the motion signal and the swallowing signal received.

The swallowing characterisation step of the method for analysing a user's swallowing by a swallowing analysis system according to the invention may comprise classifying the motion signal and swallowing signal received.

The analysis system according to the invention is of particular interest for users who have difficulty moving, in cases of movement prohibitions, and generally to limit trips of the practitioner and the users.

The invention and its different applications will be better understood upon reading the following description and upon examining the accompanying figures.

### BRIEF DESCRIPTION OF THE FIGURES

The figures are set forth by way of indicating and in no way limiting purposes of the invention.

FIG. 1 shows a schematic representation of a system for sensing swallowing of at least one user according to the invention.

FIG. 2 shows a schematic representation of a system for the remote analysis of swallowing of at least one user according to a first embodiment of the invention.

FIG. 3 shows a schematic representation of a system for the remote analysis of swallowing of at least one user according to a second embodiment of the invention.

FIG. 4 shows a schematic representation of a method for sensing swallowing of at least one user according to the invention,

FIG. 5 shows a schematic representation of a method for analysing swallowing of at least one user according to the invention.

### DETAILED DESCRIPTION

The figures are set forth by way of indicating and in no way limiting purposes of the invention.

Unless otherwise specified, a same element appearing in different figures has a single reference.

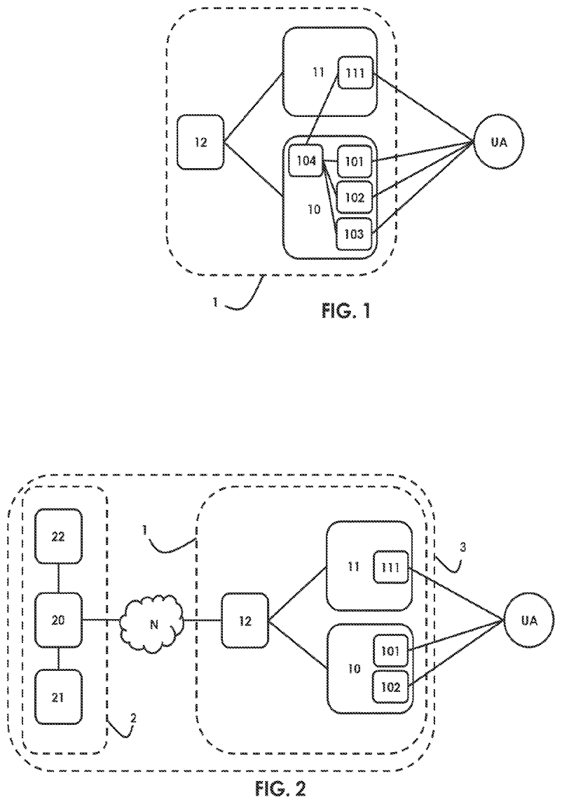

FIG. 1 shows a schematic representation of a system for sensing swallowing of at least one user according to the invention.

The sensing system 1 represented in FIG. 1 makes it possible to acquire at least one swallowing signal and at least one motion signal of a user UA. The present invention also covers cases where the sensing system 1 makes it possible to acquire at least one swallowing signal and at least one motion signal of a plurality of users (not represented).

The sensing system 1 according to the invention comprises at least one swallowing detection device 11 comprising at least one swallowing detection sensor 111 configured to acquire at least one swallowing signal of the user UA. The swallowing detection device 11 is preferably a necklace type device, worn around the neck, in a manner known to the skilled person. However, the swallowing detection device 11 may be any type of device for detecting swallowing.

The swallowing detection sensor 111 of the swallowing detection device 11 may, for example, be an accelerometer located at the larynx of the user UA. Thereby, it may measure a swallowing signal from the user UA, for example a laryngeal motion signal corresponding to swallowing or any other motion for characterising swallowing. The swallowing detection sensor 111 may, for example, be a microphone, the user swallowing signal measured thereby being a laryngeal sound, or any other sound for characterising swallowing. The swallowing detection sensor 111 may be any sensor capable of measuring a swallowing signal for characterising swallowing of the user. In addition, the swallowing detection device 11 may comprise a plurality of swallowing detection sensors 111 (not represented), for example a combination of a microphone and an accelerometer to improve the accuracy and reliability of swallowing detection.

The swallowing detection device 11 may also include at least one sensor from the heart rate, body temperature, sweating, breath sound, respiratory rate and muscle activity sensors (not represented). The swallowing detection device 11 represented in FIG. 1 may comprise all of the aforementioned sensors, but it may also comprise only one of the aforementioned sensors or any possible combination of the aforementioned sensors. The aforementioned sensors allow to know precisely the state of the user UA during a swallowing rehabilitation exercise or during a swallowing examination.

The sensing system 1 according to the invention further comprises at least one system 10 for capturing motions of the user UA configured to acquire at least one motion signal of the user UA upon sensing the at least one swallowing signal of the user UA by the sensor 111 of the swallowing detection device 11. This allows the sensing system 1 to be adaptable to existing swallowing detection devices for having, by addition in particular of the motion capture system 10, a system allowing simultaneous sensing of several types of signals, and in particular of swallowing signals and motion signals of the user UA.

The system 10 for capturing motions of the user UA according to the invention comprises at least one, preferably two, motion sensors 101 and 102 of the user UA. Thus, the system 10 for capturing motions of the user UA may comprise only the sensor 101, two sensors 101 and 102, or more than two sensors. By “motion sensor”, it is meant any sensor that can convert a user's motion into an analogue or digital signal. Thus, the sensors 101 and 102 may be cameras, or a combination of optical markers with cameras, or any other sensor that can transform a user's motion into an analogue or digital signal. Of course, a motion sensor for the purposes of the present invention also allows acquisition of position data when the user UA is not in motion. Thus, a single image from a camera allows access to position data of the user UA without any motion by the user. Depending on the system, an optical marker and two cameras can also access a position, for example by triangulation, without necessarily having any motion by the user.

The system 10 for capturing motions of the user UA according to the invention is configured to acquire at least one motion signal of the user UA upon sensing the at least one swallowing signal of the user UA by the sensor 111 of the swallowing detection device 11. By “motion signal” of the user UA, it is meant any signal for restoring a motion, a posture or a change in posture of the user UA, that is any signal representing a motion, a posture or a change in posture of the user UA, such as an image or a sequence of images (video), a two- or three-dimensional representation of particular points or limbs or set of limbs of the user UA, an electrical signal proportional to a quantity measured, or any other signal allowing representation or restoration of motion(s), posture or a change in posture of the user UA.

By user “motion”, it is meant any change in position of the user UA and more specifically any change in position of a limb or set of limbs of the user relative to another or relative to a reference frame, for example any change in position of the head relative to the neck and/or relative to the thorax and/or relative to a fixed or moving reference frame. Furthermore, a signal indicating that no motion has occurred is a motion signal, as the information that no motion has occurred is important information regarding the motions of the user UA. In particular, any succession of images of the user UA's head, neck and thorax assembly or any combination of these three limbs or sets of limbs is included in the term “motion signal” of the user UA. Preferably, the system 10 for capturing motions of the user UA makes it possible to capture a change in posture of the user UA.

The system 10 for capturing motions of the user UA thus comprises at least one sensor 101, for example a camera. The system 10 for capturing motions of the user UA may further comprise a second sensor 102, for example another camera, configured to acquire images viewed from another angle relative to the first camera 101. For example, the camera 101 is configured to acquire frontal images of the user UA and the camera 102 is configured to obtain profile images of the user UA. When reference is made to a camera being “configured to acquire” particular images, it is meant that the camera is oriented and/or positioned and/or set to capture those particular images. In another embodiment, the sensor 101 may be a camera and the sensor 102 may be an infrared sensor or an infrared projector in cooperation with the camera 101. The sensors 101 and 102 may be any type of sensor for capturing a signal of motion of the user UA, preferably motion at the larynx or head-neck-thorax assembly. When the sensors 101 and 102 are cameras, or when at least one of the sensors 101 or 102 is a camera, the system 10 for capturing motions of the user UA according to the invention may implement adaptive blurring of the face of the user UA in order to maintain the anonymity of the user UA.

Preferably, the system 10 for capturing motions of the user UA comprises a motion capture marker 103 in cooperation with at least one of the two sensors 101 or 102, preferably in cooperation with both sensors 101 and 102. Such a motion capture marker 103 allows the user's motions to be captured more accurately than by using cameras alone and by performing pattern or motion recognition from the images captured. The motion capture marker 103 is worn by the user UA, either directly bonded, attached or hooked to their skin or a garment they are wearing, or bonded, attached or hooked to the swallowing detection device 10 they are wearing. A motion capture marker 103 may, for example, be a passive optical marker, reflecting, for example, infrared radiation emitted by the cameras 101 and 102 and back captured by the cameras 101 and 102. A single camera 101 may be used, but the invention is preferably implemented with two cameras 101 and 102. A motion capture marker 103 may also, for example, be an active optical marker, emitting light or infrared radiation towards cameras or light sensitive sensors. The motion capture marker 103 may furthermore be any type of marker that allows capture of motions of the user UA.

The motion signal acquired by the motion capture system 10 is acquired upon sensing the at least one user swallowing signal by the sensor 111 of the swallowing detection device 11. For this, the motion capture system 10 comprises at least one processor 104 configured to synchronise signal sensing of both types. In this way, it is possible to relate motions of the user UA to their swallowing. For example, the processor 104 may be configured to transmit a motion measurement command to the sensors 101 and 102 of the motion capture system 10 when it detects that the swallowing detection device 11 is measuring a swallowing signal of the user UA. For this, the processor 104 may be directly connected to the swallowing detection sensor 111. The processor implements instructions stored by a memory of the motion capture system 10 (not represented) which, when executed by the processor 104, cause it to implement synchronisation of the measurement of the swallowing and motion signals of the user UA. In another embodiment not represented, the processor may not be an integral part of the motion capture system 10, but may for example be part of the swallowing detection device 11, or may be external to the swallowing detection device 11 and the motion capture system 10. Preferably, the processor 104 is included in the motion capture system 10 so that the motion capture system 10 can simply be added to an existing swallowing detection device 11 to improve swallowing detection and analysis.

The swallowing sensing system 1 according to the invention may further comprise a network module 12, as represented in FIG. 1. This network module 12 may enable the swallowing and motion signals to be sent through a network to another analysis device described later. The invention also covers swallowing sensing systems 1 not comprising a network module 12. Advantageously, the network module 12 allows signals captured by the motion capture system 10 and the swallowing detection device 11 to be analysed remotely. The network module 12 may allow communication through one or several networks, of the same or different types. For example, the network module 12 may allow communication through an Internet network, for example using the TCP/IP protocol suite. The network module 12 may allow communication via a wireless connection, for example Wi-Fi, or via a wired connection, for example the Ethernet.

The FIG. 2 shows a schematic representation of a system for the remote analysis of swallowing of at least one user according to a first embodiment of a second aspect of the invention.

Indeed, a second aspect of the invention relates to a system for the remote analysis 3 of swallowing of a user UA, the remote analysis system 3 comprising the system for sensing 1 swallowing of a user UA as previously set forth and a remote analysis device 2. The swallowing sensing system 1 of a user UA and the remote analysis device 2 are connected to each other and communicate through a network N. As previously indicated, this network N can be any communication network, preferably the Internet. To communicate across the network N, the swallowing sensing system 1 of a user UA comprises a network module 12 and the remote analysis device 2 comprises a network module 20. The two network modules 12 and 20 may be the same or different, and each has to be capable of enabling communication across the network N. The network module 20 may be capable of communicating across one or more networks, of the same or different types. For example, the network module 20 may enable communication across an Internet network, for example using the TCP/IP protocol suite. The network module 20 may allow communication via a wireless connection, for example Wi-Fi, or via a wired connection, for example the Ethernet. The network module 20 is configured to receive from the network module 12 of the sensing system 1, via the network N, the at least one swallowing signal of the user UA measured by the sensor 111 of the swallowing detection device 11 and the at least one user motion signal sensed by the motion capture system 10 upon sensing the at least one swallowing signal of the user UA. Thus, the remote analysis device jointly receives both signals measured during the same time period, allowing analysis of both signals to characterise swallowing of the user UA.

The remote analysis device 2 further comprises a display module 21 configured to simultaneously display the at least one swallowing signal of the user UA and the at least one motion signal of the user UA upon sensing the at least one swallowing signal of the user UA. The display module 21 may be a screen or any other type of device for displaying data to a user of the remote analysis device 2 such as a practitioner.

The remote analysis device 2, in a preferred embodiment and as represented in FIG. 2, further comprises a processor 22, configured to characterise swallowing of the user UA from signals received from the swallowing sensing system 1. By “characterising”, it is meant giving an attribute to swallowing represented by a signal. By “characterising” a signal, it is meant associating an attribute of swallowing with a signal representing that swallowing. An example of characterising, which is also a preferred embodiment, is classifying a signal, for example into a class corresponding to correct swallowing or into a class corresponding to incorrect swallowing. This classification can be performed in a manner known to the skilled person by an automatic learning algorithm using a statistical model or a neural network. Signals from the sensor 111 may allow the classification model to be more accurate and reliable in its decision making to characterise a swallowing signal as being “incorrect”, that is representative of a dysphagia of the user under study, for example a swallowing presenting a risk of false passage or pulmonary aspiration, or “correct”, that is representative of swallowing not characteristic of a dysphagia of the user under study. The processor 22 may take account only of the swallowing signals from the sensor 111 of the swallowing detection device 11, or may take account both of the swallowing signals from the sensor 111 of the swallowing detection device 11 and the motion signals from the motion capture system 10. In particular, this may allow for a better characterisation of swallowing.

Preferably, the swallowing signals, the motion signals and their characterisation assigned by the processor 22 are displayed together by the display module 21. Even more preferably, the swallowing and motion signals received may be displayed in real time or near real time, that is, for example, as soon as they are received by the receiving module 20, after characterising at least one of these signals by the processor 22. The swallowing and motion signals received may be displayed without being characterised, for example before the characterisation has been performed by the processor 22, and the characterisation may be displayed subsequently by the display module 21, when available, that is when it has been performed by the processor 22. Even more preferably, the remote analysis device comprises a storage module, configured to store the swallowing and motion signals received and the characterisation of swallowing represented by these signals. The storage module may also be accessible through a network.

FIG. 3 shows a schematic representation of a system for remotely analysing swallowing of at least one user according to a second embodiment of the invention.

In the second embodiment represented in FIG. 3, the remote analysis device does not include a processor 22. The swallowing sensing system 1 comprises the processor 22. The characterisation of the swallowing signals and/or motion signals is thereby performed at the sensing system 1. Thus, only the swallowing characterisation and motion signals can be sent to the remote analysis device 2 through the network N, for analysis by a practitioner or a user through the display module 21. Alternatively, the swallowing detection signal may be sent, together with the motion signal and the swallowing characterisation, or any possible and interesting combination of these three pieces of information. Preferably, the user of the remote analysis device 2 can visually analyse through the display module 2 the swallowing and motion signals and the associated swallowing characterisation together, that is on the same screen or the same display.

Thus, a user of the remote analysis device 2, for example a practitioner, can perform remote consultations (also called “teleconsultations”), having access to a maximum of information and in particular to images of the patient or the user UA swallowing together with the corresponding swallowing signals.

The remote analysis device 2 may further allow the swallowing and motion signals received to be analysed in a more advanced manner than by simply displaying, for example by using signal analysis and processing and/or image analysis and processing tools. For this, the remote analysis device 2 may further comprise a human-machine interface (not represented).

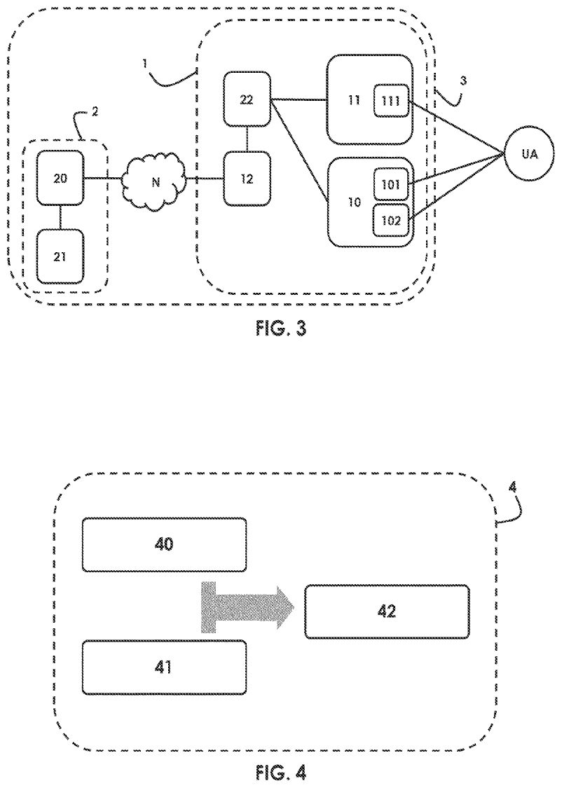

FIG. 4 shows a schematic representation of a method for sensing swallowing of at least one user by a swallowing sensing system according to the invention.

The method 4 for sensing swallowing of at least one user UA comprises three steps 40 to 42.

In a first step 40 at least one swallowing signal is sensed by a swallowing detection device 11 of the swallowing sensing system 1 as set forth previously. This sensing step comprises measuring, by a swallowing sensor 111 of the swallowing sensing device 11 as set forth above, at least one signal associated with swallowing, for example sound or accelerometery.

A second step 41 comprises acquiring, by the motion capture system 10, a motion signal of the user UA simultaneously with sensing the swallowing signal by the swallowing detection device. As explained previously, the synchronisation of steps 40 and 41 may be achieved using a processor 104, or by any other means that allows synchronisation of measurement of both signals.

The method 4 for sensing swallowing of at least one user UA preferably comprises a step 42 of sending the swallowing signal and the motion signal through a network N. This sending is performed by the network module 12 of the swallowing sensing system 1.

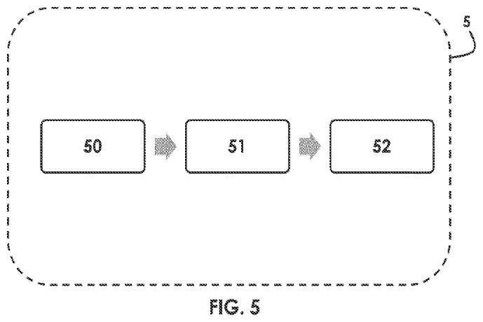

FIG. 5 shows a schematic representation of a method for remotely analysing swallowing of at least one user by a swallowing analysis system according to the invention.

The method 5 for remotely analysing swallowing of at least one user UA by the swallowing analysis system 3 as previously set forth comprises three steps 50 to 52.

In a first step 50, the remote analysis device 2 receives, through the network N, the at least one swallowing signal and the at least one motion signal of the user UA simultaneously acquired as described in the method 4 for sensing swallowing.

The method 5 for remotely analysing swallowing then comprises a second step 51 of characterising swallowing by the processor 22 of the swallowing analysis system 2 from the motion signal and swallowing signal received. As previously described, this step 51 of characterising the swallowing signal and motion signal received may include, for example, classifying the motion signal and swallowing signal received.

The method 5 for remotely analysing swallowing preferably includes a third step of displaying, by the display module 21, the swallowing and motion signals received and the characterisation performed in step 51 of the method 5. As previously explained, displaying the characterisation may include displaying the attribute given to the signals or swallowing by the processor 22 in the characterisation step 51.

1. A system for sensing swallowing of at least one user, the system comprising:
at least one swallowing detection device comprising at least one swallowing detection sensor configured to acquire at least one user swallowing signal,
at least one user motion capture system configured to acquire at least one user motion signal upon sensing the at least one user swallowing signal by the sensor of the swallowing detection device, the motion capture system comprising two cameras, one of the two cameras being configured to acquire frontal images of the user and another of the two cameras being configured to obtain profile images of the user, and
a processor configured to characterise the user's swallowing from the user swallowing signal and the user motion signal.

2. The swallowing sensing system of claim 1, wherein the motion capture system further comprises at least one motion capture marker in cooperation with the at least one camera.

3. The swallowing sensing system according to claim 1, wherein the at least one camera is configured to acquire images of the head, neck and thorax of the user.

4. The swallowing sensing system according to claim 1, wherein the swallowing detection sensor is a microphone for detecting swallowing sound and/or an accelerometer to detect swallowing vibration.

5. The swallowing detection system according to claim 1, wherein the swallowing detection device further comprises at least one sensor from heart rate, body temperature, sweating, breath sound, respiratory rate and muscle activity sensors.

6. The swallowing sensing system according to claim 1, further comprising a network module configured to send through a network the at least one user swallowing signal and the at least one user motion signal upon sensing the at least one user swallowing signal.

7. A system for analysing swallowing of at least one user comprising:
the user swallowing sensing system according to claim 6 and
a remote analysis device comprising:
a network module configured to receive from the network module of the sensing system, via the network, the at least one user swallowing signal and the at least one user motion signal upon sensing the at least one user swallowing signal, and
a display module configured to simultaneously display the at least one user swallowing signal and the at least one user motion signal upon sensing the at least one user swallowing signal.

8. The swallowing analysis system according to claim 7, wherein the remote analysis device comprises a processor configured to characterise the user's swallowing from the user swallowing signal.

9. The swallowing analysis system of claim 8, wherein characterising swallowing by the processor comprises classifying the swallowing signal.

10. The swallowing analysis system according to claim 7, wherein the processor is configured to characterise swallowing of the user by further using the user motion signal.

11. A method for sensing swallowing of at least one user by a swallowing sensing system according to claim 1, the method comprising:
sensing at least one swallowing signal by a swallowing detection device of the swallowing sensing system;
acquiring, by the motion capture system, the user motion signal simultaneously with sensing the swallowing signal by the swallowing detection device.

12. A method for analysing swallowing of at least one user by a swallowing analysis system according to claim 8, the method comprising:
receiving, by the remote analysis device, the at least one swallowing signal and the at least one user motion signal acquired simultaneously with sensing the swallowing signal, and
characterising swallowing by the processor of the swallowing analysis system from the motion signal and swallowing signal received.

13. A method for analysing swallowing according to claim 12, wherein characterising swallowing comprises classifying the motion signal and swallowing signal received.
